(19) United States
(12) Patent Application Publication
(10) Pub. No.: US 2024/0268791 A1
(43) Pub. Date: Aug. 15, 2024

(54) PORTABLE IMAGING SYSTEM

(71) Applicants: SORBONNE UNIVERSITE, Paris (FR); CENTRE NATIONAL DE LA RECHERCHE SCIENTIFIQUE, Paris (FR); INSERM (INSTITUT NATIONAL DE LA SANTÉ ET DE LA RECHERCHE MEDICALE), Paris (FR)

(72) Inventors: Abderrahmanne AISSANI, Paris (FR); Arthur CHAVIGNON, Gentilly (FR); Antoine COUDERT, Paris (FR); Olivier COUTURE, Issy-les-Moulineaux (FR); Baptiste HEILES, Den Haag (NL); Vincent HINGOT, Paris (FR)

(73) Assignees: SORBONNE UNIVERSITE, Paris (FR); CENTRE NATIONAL DE LA RECHERCHE SCIENTIFIQUE, Paris (FR); INSERM (INSTITUT NATIONAL DE LA SANTÉ ET DE LA RECHERCHE MEDICALE), Paris (FR)

(21) Appl. No.: 18/566,843

(22) PCT Filed: Jun. 3, 2022
(86) PCT No.: PCT/EP2022/065259

§ 371 Date: Dec. 4, 2023

(30) Foreign Application Priority Data
Jun. 4, 2021 (EP) 21305759.9

(51) Int. Cl. A61B 8/08; A61B 8/00

(52) U.S. Cl. CPC A61B 8/481; A61B 8/0891; A61B 8/4209; A61B 8/4245; A61B 8/4427; A61B 8/462

(57) ABSTRACT

A portable imaging system for visualizing a biological target structure by a tracking element introduced therein, including: a control unit with a memory for storing at least one ultrasound image of the target structure, a probe to be removably fixed to a biological fixing structure surrounding at least partially the target structure, the probe being connected to the control unit, and a positioning assistance module of the probe. The probe is capable of detecting the tracking element by means of ultrasound, the control unit being able to locate it, in real time, inside the target structure, so as to characterize it geometrically and create the ultrasound image. The control unit can also display it on a display device. The ultrasound image allows rapid diagnosis to be made independently of any location and environment.

### TECHNICAL FIELD

The present invention relates to the field of ultrasonic medical imaging.

### STATE OF THE ART

As is well known per se, echography consists of transmitting and receiving ultrasound waves through the biological tissues and structures of a patient and using the echoes obtained in these biological tissues and structures to reconstruct an ultrasound image. The term image applies to all representations obtained from processing echoes and can be 1D (in the form, for example, of a table of values or a score), 2D or 3D. Ultrasounds can be used, for example, to measure the echogenicity of tissues. An ultrasound image can therefore be a succession of information such as the positions of tracking elements.

In addition, the introduction of a tracking element, for example a contrast agent, makes it possible to enhance the echoes, and therefore the perceived signal, of all the vessels and thus obtain an image of tissue perfusion. A contrast agent that is well known in the art can take the form of an intravenously injectable microbubble solution. In general, a series of microbubbles is injected at a time. Typically, several tens or hundreds of millions are injected into the human vascular network.

As ultrasound is traditionally based on a wave-based approach, it is limited by diffraction, i.e. the used wavelength limits the precision with which biological tissues and structures can be characterised. Paradoxically, short wavelengths, which are conducive to greater resolution, are attenuated more strongly in tissues than longer wavelengths.

### AIM OF THE INVENTION

Super-resolved ultrasound or Ultrasound Localization microscopy abbreviated by the English acronym “ULM” or the French acronym “MLU” for microscopy by ultrasonic localization (Couture et al. IEEE UFFC 2018) is a technique that circumvents the rules of diffraction using the aforementioned microbubbles. By taking multiple images of a tissue containing vessels with microbubbles flowing through them, the position of each microbubble can be determined with a resolution more precise than wavelength. By accumulating the super-resolved position of each microbubble, it is possible to create an image of the vessels whose resolution exceeds the theoretical diffraction limit. Until recently, ultrasound scanners used very little post-processing and the important signal processing steps were carried out in an analogue way. Recently, computer components have made it possible to process large volumes of data simultaneously and to work on signals that have been digitised and sent to a computer. Ultrasound super resolution (ULM) is a digital-only imaging method that improves resolution by a factor of 10 compared with conventional methods. It is based on various stages carried out sequentially using a device that records ultrasound echoes and processes them on a computer.

This method is currently carried out sequentially in the following stages:
1) an ultrasound signal transmission/reception sequence defined by software: these are referred to as Radio Frequency (RF) signals, and correspond to N time recordings made by N ultrasound sensors. Each transmission/reception sequence is repeated at a high rate (e.g. 500 Hz),
2) a beamforming step to convert these time signals into an ultrasound image, more precisely, an ultrasound image characterising the medium,
3) a step to separate the microbubble echoes from the echoes coming from the other tissues,
4) a detection step to identify each microbubble individually,
5) a sub-pixel localisation step consisting of determining the position of a microbubble in the image with an accuracy lower than the original dimensions of the image,
6) a tracking step, which links the positions of the same microbubble between successive images to form trajectories of the path of the microbubbles in the bloodstream,
7) a trajectory processing step to correct spatial and temporal sampling artefacts,
8) a display step, enabling reconstruction of all the vascularisation present in the fields of view,
9) a step for correcting the aberration of ultrasound echoes which are distorted as they pass through a bone (e.g. the skull bone), which may be carried out in parallel with or upstream of the processing steps,
10) a motion correction step which can be performed in parallel with the processing steps and which estimates the displacements between successive images so as to correct them.

The size of the ultrasound image depends on the geometry of the probe used. As is well known, a 3D image can be obtained using so-called matrix probes whose sensors are arranged in a plane.

Medical imaging is an essential assistance (aid) in the establishment of many diagnoses, particularly stroke-related diagnoses. However, in emergency cases, the imaging required for diagnosis can often not be carried out quickly enough, either because the patient's transport time is too long, or because the care centre's imaging equipment was already being used for another diagnosis on another patient. The key point of the problem currently encountered by medical imaging is the lack of diagnostic possibilities outside the hospital. It has been identified that, technically, this is limited by the inadequacy of imaging techniques, particularly conventional ultrasound-based techniques. There is therefore a real need for portable medical imaging equipment that can be brought to the patient, without necessarily having to first bring the patient to the medical imaging equipment. However, regardless of whether or not it is possible to create a medical image independently of the location of a patient potentially needing it, it must then be possible to read and interpret it, and this may be impossible if there is no experienced practitioner nearby. There is therefore a real need for a system that can create medical images that can be interpreted and read by an experienced practitioner regardless of where the patient and practitioner are located.

One of the aims of the present invention is to propose the use of the ultrasound technology described above, enabling ULM-type super-resolution, in the management of patients needing a diagnosis using such a technology, regardless of the location of the patient and the practitioner having to establish the diagnosis resulting from this technology. More specifically, the aim of the present invention is to provide new means of assisting diagnosis on the move (in particular by obtaining images). New means facilitating portable diagnosis are made possible by the use of super-resolved ultrasound (SRU) and by the fact that SRU makes it possible to implement the various parts of the invention described below.

### DESCRIPTION OF THE INVENTION

This objective is achieved by means of a portable ULM imaging system for imaging a biological target structure of a patient by means of at least one tracking element introduced within said biological target structure, the portable ULM imaging system comprising:
a control unit with a memory, the memory being intended to store at least one ultrasound image characterising the biological target structure,
at least one first probe intended to be removably fixed to a biological securing structure of the patient, the biological securing structure at least partially surrounding the biological target structure, the at least one first probe being connected to the control unit,
a positioning assisting module for the at least one probe,
   the at least one first probe enabling the system to detect the at least one tracking element by means of ultrasounds, the control unit being configured to locate, in real time, the at least one tracking element inside the biological target structure, so as to implement a ULM method to characterise the biological target structure and create the ultrasound image,
   the control unit further being designed to display, on a display device, the at least one ultrasound image stored in the memory,
   the ultrasound image enabling an operator to make a rapid diagnosis independently of the patient's location and environment.

Thus, thanks to the ULM, this solution makes it possible to achieve the aforementioned objective. In particular, any person requiring a diagnosis based on medical imaging can be diagnosed independently of where they are located and independently of where the practitioner able to make the diagnosis is located. The image created, regardless of its form, has a strong diagnostic assistance (aid) value. Its use in mobility and by non-experts is enabled by the combinations and synergies between super-resolved ultrasound (ULM) and the different elements of the system according to the invention which make it possible to achieve good diagnostic quality.

The implementation of ULM on such a portable system allows, thanks to the gain in resolution that it allows compared to conventional technologies, to carry out a diagnosis on a portable system, where conventional portable systems would be incapable of doing so. This also makes it possible to embark several modules whose operation is permitted by the ULM, and which facilitate use in mobility and by non-expert operators.

The system according to the invention may comprise one or more of the following characteristics, taken in isolation from each other or in combination with each other:
the biological target structure can be the cerebral blood system and the ultrasound image may be an angiography,
the at least one tracking element may be a contrast agent,
the display device may be located in a different place from the control unit and/or the at least first probe,
the control unit and the display device may communicate by electromagnetic waves,
the system may comprise a diagnostic assistance module,
the diagnostic assistance module may enable a first operator at the patient's bedside to be put into contact with a second operator able to read the ultrasound image displayed on the display device,
the diagnostic assistance module may comprise software for assisting an operator at the patient's bedside,
the positioning assisting module may be a passive mechanical module,
the positioning assisting module may be an active electronic component,
the positioning assisting module may calculate a transmission quality index for each probe,
the position assisting module may send at least one feedback signal so as to help maximise the quality index,
the system may comprise a transmission module.

A further object of the present invention is about a rapid imaging method intended to visualise a biological target structure of a patient, the method enabling the system according to any one of the preceding claims to be implemented, the method comprising the following steps, in the chronological order in which they are stated:
a) connecting, if necessary, by a first operator, of the at least one probe to the control unit,
b) engaging the positioning assistance module,
c) positioning the probes on the patient's biological securing structure,
d) acquisition of a first ultrasound image,
e) correction, if necessary, of the positioning of at least one probe by means of the positioning assistance module, until optimisation of the signal perceived by the control unit,
f) positioning of the at least one probe on the biological securing structure,
g) evaluation of the quality of the signals before launching the sequence for acquiring a new ultrasound image,
h) injection, in the first few seconds of acquisition, of tracking elements into the patient's biological target structure,
i) acquisition of the ultrasound image,
j) correction, if necessary, of the positioning of at least one probe by means of the positioning assistance module until optimisation of the signal perceived by the control unit using ULM technology,
k) creation of the ultrasound image of the biological target structure using ULM technology,
l) immediate transmission of the ultrasound image to a display device within reach of a second operator.

After the step of positioning the at least one probe, the method may include a step of fixing the at least one probe to the biological fixing structure.

### BRIEF DESCRIPTION OF THE FIGURES

The invention will be better understood, and other purposes, details, characteristics and advantages thereof will become clearer on reading the following detailed explanatory description of embodiments of the invention given by way of purely illustrative and non-limiting examples, with reference to the appended schematic drawings.

In these drawings

FIG. 1 is a schematic illustration of a portable imaging system according to the invention,

FIG. 2 is an illustration of an ultrasound image obtained by means of the system according to the present invention,

FIG. 3a is a schematic illustration of an ultrasound probe of the echographic type used in the system according to the invention,

FIG. 3b is a schematic illustration of an angled ultrasound probe used in the system according to the invention.

### DETAILED DESCRIPTION

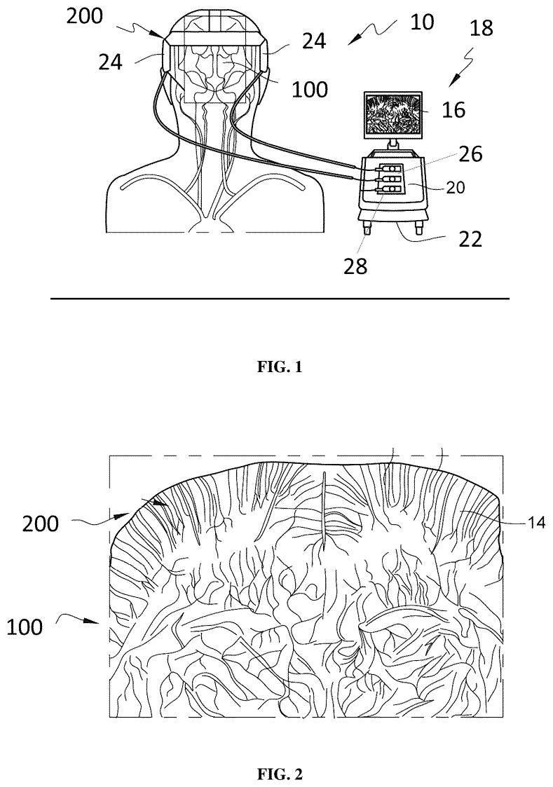

As can be seen in FIG. 1, the present invention relates to a portable imaging system 10 intended to visualise a biological target structure 100 of a patient, such as for example a cerebral vascularisation, and to locate a tracking element within said biological target structure 100, so as to be able to create an ultrasound image 16 readable on a display device 18, for example a screen.

In a preferred embodiment, the biological target structure 100 is the cerebral blood system and the ultrasound image is an angiography.

According to a particular embodiment, the ultrasound image 16 is in 1D, for example in the form of a table storing the positions of the tracking elements 14 (for example microbubbles) or a score. The image may also be 2D.

In another embodiment, the ultrasound image 16 is 3D. This makes it possible to acquire an entire volume of the patient's biological target structure 100 with each injection of tracking elements 14, rather than reconstructing the biological target structure 100 plane-by-plane, which would require numerous injections of tracking elements 14. More generally, the 3D aspect frees up issues of operator dependency. 2D imaging requires an operator to select a plane, whereas a 3D volume enables everything to be acquired and then assessed retrospectively. The use of a 3D ultrasound image offers an additional synergy that can be exploited: the use of super-resolution (ULM) combined with 3D limits the dependence on the operator, and therefore makes mobile diagnosis more reliable without an expert operator.

Alternatively, the entire volume of the biological target structure 100 can be reconstituted from a series of small partial 3D volumes of the biological structure. This ultrasound image 16 is intended to enable rapid diagnosis, particularly in the case of a cerebrovascular accident. More specifically, ultrasound image 16 enables an operator to make a rapid diagnosis independently of the patient's location and environment. It is therefore possible to diagnose a patient at home or, for example, in an ambulance. In the present invention, the notion of “rapid” is defined in relation to an ambulance journey: the diagnosis must be able to be made during an ambulance journey. The term “rapid” therefore refers to a time of several seconds to around twenty minutes, and should be compared with current diagnostic times, which can be several hours.

Thus, as can be seen from FIG. 1, the portable imaging system 10 according to the invention comprises:
a control unit 20 with a memory 22, said memory 22 being locatable anywhere in the system 10,
at least one first probe 24 intended to be removably fixed to a biological securing structure 200 of the patient, the biological securing structure 200 at least partially surrounding the biological target structure 100, the at least one first probe 24 being connected to the control unit 20,
a position assisting module 26 for the at least one probe 24.

In certain embodiments, the portable imaging system 10 according to system 10 comprises a diagnostic assistance module 28.

By portable it is meant a system that can be moved simply by the physical strength of one or more operators, without the need for electronic tools or transport tools such as trolleys or dollies. By portable we mean a system that can be moved easily by being carried by a user, for example by means of handles or straps. Ideally, the system weighs between 10 and 30 kilograms.

More particularly, the portability (portable aspect) of the system 10 according to the present invention is made possible by the implementation of the ULM as well as by the various synergies offered by the ULM, i.e.:
telemedicine thanks to lighter data,
the ability to adapt probe placement automatically on the basis of ULM data
the ability to calculate specific biomarkers from ULM data (see later in the description for specific examples).

It is from the synergy between the various parts of the present invention that the portability of system 10 derives, in the sense that its use makes it possible to carry out a diagnosis anywhere and without being dependent on an operator who is an expert in ULM technology.

In a preferred embodiment, system 10 has a volume of less than or equal to 1m3 and a weight of less than or equal to 15 kg.

The tracking element 14 is intended to be introduced into the biological target structure 100, for example by injection. In the case where this tracking element 14 is a contrast agent, more particularly a microbubble, millions of tracking elements 14 are thus intended to be injected into the patient. They are all intended to be located within the biological target structure 100 by means of at least one probe 24 and thus make it possible to delimit the said biological target structure and create an ultrasound image 16 that can be used using ULM technology.

### Control Unit

The control unit 20 is designed to produce and store at least one ultrasound image 16 of the patient's biological target structure. It thus comprises:
a digital ultrasound scanner, the main components of which are:
   an electronic acquisition card and an electronic emission card, or an electronic acquisition/emission card,
   computer components (processors, graphic card, etc.),
   a standard medical box incorporating the above components,
software that performs all of the following tasks:
   interfacing with the electronic card(s),
   controlling the transmission and reception of ultrasound signals,
   storing digitised signals in the memory,
   forming channels to prepare the data for the processing stage,
   a processing software step enabling ultrasound super-resolution,
   interfacing with the operator.

The memory 22, which can be located anywhere in the system 10, of the control unit 20 is thus intended to store the at least one ultrasound image 16 of the biological target structure 100. An example of such an ultrasound image 16 2D is visible in FIG. 2.

The control unit 20 is further designed to display, on the display device 18, the at least one ultrasound image 16 stored in the memory 22. The control unit 20 thus makes it possible to display (directly or indirectly) the location of the at least one tracking element 14 in the at least one ultrasound image 16.

The control unit 20 also comprises a transmission module, so as to enable the data collected by the probes 24 (ULM data, for example) to be transmitted remotely, so as to enable the ultrasound image 16 to be displayed on a display device 18 located at a location other than the control unit 20, thus enabling another operator to establish a remote diagnosis.

The present invention, which seeks to produce and present means, in particular in the form of an ultrasound image, making it possible to establish a diagnosis on the move by combining super-resolved ultrasound (SRU) and telemedicine, is, by definition, linked to the concept of ambulatory telemedicine (whether urgent or not) on the basis of rapid imaging of a biological target structure 100 of a patient. In particular, the present invention enables ULM angiography of the brain and the placement, Doppler and ULM data, after automatic processing, can thus preferably be transmitted to an operator (e.g. a radiologist) remotely. As this transmission takes place after the data has been processed by computer, for example the ULM processing, the quantity of data to be transmitted is greatly reduced and can therefore be transmitted quickly and easily, allowing mobility. In fact, an entire volume of ultrafast 3D ultrasound data for a few minutes of acquisition can be of the order of a Terabyte. The position of each of the tracking elements 14 (e.g. microbubbles), even if they number in the millions, is very parsimonious data which can be transmitted more easily (Megabytes). Following acquisition at the patient's bedside, each vectorised ultrasound image 16 is securely transmitted remotely to the second operator, who can then make a diagnosis. The diagnosis is then used to establish whether treatment (e.g. thrombolysis) needs to be carried out on site. The raw data is saved locally in memory 22 of the control unit 20, for analysis and archiving, for example on arrival at the hospital.

### The at Least First Probe

In the embodiment illustrated in FIG. 1, the system 10 comprises two probes 24. These are ultrasonic probes. Each probe 24 thus comprises at least one ultrasonic sensor. Generally speaking, and as is well known per se, ultrasonic probes are usually designed so that they can be held and handled easily by hand. This imposes a quasi-cylindrical shape where the cables transmitting the electrical signals between each probe 24 and the control unit 20 are perpendicular to the plane containing the at least one ultrasonic sensor. Each probe 24 is thus capable of making the ultrasound-electronic conversion and thus enabling the system 10 to detect each tracking element by means of ultrasound. More particularly, each probe 24 is spatially sampled in two dimensions to be able to reconstruct an entire volume.

Each probe 24 may be a matrix probe, more particularly a multiplexed probe. In this case, as a minimum, each probe 24 must be provided with at least several transducers in two dimensions. In some cases, each probe 23 may be provided with a Cartesian grid of transducers.

Thus, the ultrasound image 16 consists of three-dimensional data obtained using a probe 24 comprising transducers positioned in at least two dimensions (matrix of elements).

As each probe 24 is connected to the control unit 20, the control unit 20 can thus locate, in real time, each tracking element within the biological target structure 100. This makes it possible to detail and geometrically characterise the biological target structure 100 and to create an ultrasound image 16 thereof.

Each probe 24 may be attached to the patient's biological securing structure 200 by means, for example, of a helmet (in the case where the biological attachment structure 200 is the patient's skull) or a collar fitted with one or two probes 24, or even held in the hand. In an alternative embodiment, the patient can be seated on a seat where the probes 24 are attached.

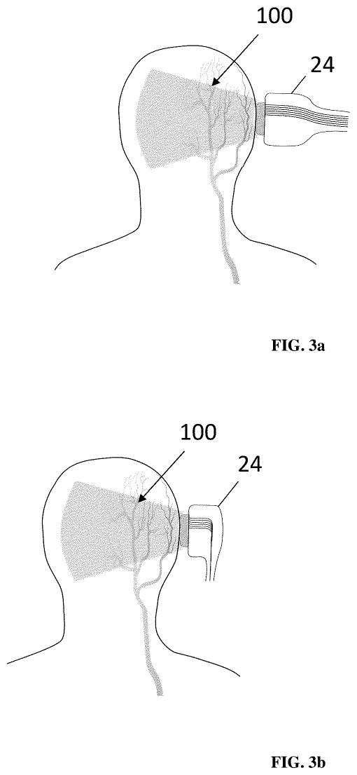

Each probe 24 can be designed as shown in FIG. 3, i.e. with the cables positioned in the plane of the ultrasonic sensors. In this way, it can be held in the palm of the operator's hand, making it easier to maintain a stable position for a longer period of time.

In the case where the biological target structure 100 is the cerebral blood system, the biological securing structure 200 is the patient's skull.

### Position Assisting Module of the at Least One Probe

The position assisting module 26 is a mechanical and/or electronic system for assisted placement of each probe 24 on the biological securing structure 200.

Super-resolved echography differs from standard echography in that it is carried out by imaging the same location for long periods. The acquisition period can last from a few seconds to several minutes. In fact, to collect the super-resolved position of thousands of tracking elements such as contrast agents, more particularly microbubbles, while avoiding their respective signals interfering, it is necessary to make thousands of images of the same biological target structure 100 of the patient. In contrast, standard ultrasound is real-time and is performed by scanning the organs with two-dimensional imaging and identifying the planes of diagnostic interest.

It is therefore essential that each probe 24 is placed in contact with the biological securing structure 200 so that the emission zone of each ultrasound beam emitted by each probe 24 allows the highest possible transmission quality index to be obtained. To enable the system 10 to function correctly, it is therefore necessary to ensure that each probe 24 is placed in the right place.

The aim of the position assisting module 26 is to assist a non-expert operator to position each probe 24 correctly.

In a first embodiment, the position assisting module 26 is a passive mechanical module, allowing correct positioning of each probe 24 by means, for example, of a complementary shape with a part of the patient's anatomy. The position assisting module 26 can thus comprise a part or part of a part forming a negative of an anatomical part of a patient, for example the ear.

In a second embodiment, the position assisting module 26 is an active electronic module.

This active embodiment involves making acquisitions to characterise the quality of the ultrasound transmission and identify the ideal position in which the ultrasound emission should be carried out and therefore the ideal position in which each probe 24 should be fixed to the biological securing structure 200 of the patient. The quality of the ultrasound transmission is determined from a quality index calculated from one or a combination of the following elements:
low-resolution ultrasound imaging of the biological target structure 100 using imaging methods already known in the art (e.g. pulsed Doppler imaging),
recognition of the environment imaged with a 2D probe,
the distortion analysis of the echoes received with regards to a propagation in an ideal medium, for example by measuring the deviation from the spatial impulse response or the coherence function of the ultrasonic echo, the distortion being a measurement carried out at using tracking elements 14 and can be directly calculated on ULM data,
the analysis of the signals diffused on the low-resolution ultrasound imaging carried out according to imaging modes already known to the state of the art, of intrinsic properties of the biological securing structure 200 (for example the thickness of the ‘skull bone).

The emission zone of each ultrasonic beam is then determined so that the transmission quality index is as high as possible. Beam steering can be done using one or a combination of the following methods:
by a physical movement of each probe 24 which can be carried out manually by the operator thanks to visual and/or auditory sensory indicators,
in a motorized and/or automated manner using a mechanical device,
electronically and/or automated using an acoustic device making it possible to direct the ultrasound beam by electronic control of the emission.

By “automated” is meant an electronic displacement by changing the phases or elements of each probe 24 used. For example, this could be a very large probe 24 with many elements of which only a fraction are used to reconstruct the final image. Positioning would therefore be the step for establishing the best part of the probe 24 to use.

Thus, once a first quality index is calculated by the position assisting module 26 for a first positioning of the probes 24, a feedback signal is sent by the position assisting module 26 indicating the quality of the first positioning of each probe 24. If the quality of this first positioning is not good enough, the position assisting module also indicates a direction of movement for the probe 24, to improve the quality index. This may be a gradual signal to enable very precise positioning. The signal can also take into account the movement history of each probe 24. The probe 24 is then moved either manually or automatically and a next quality index is calculated. This is repeated until the quality index is satisfactory.

These positioning steps can also be carried out during imaging to avoid spatial drift of the ultrasound probe.

The position assisting module 26 for the at least one probe 24 may form part of the control unit 20.

### Diagnostic Assistance Module

According to a first embodiment, the diagnostic assistance module 28 allows the assistance of a first operator located at the patient's bedside, by a second operator, preferably an expert located in a different location. The diagnostic assistance module 28 operates using telecommunications technologies and is, like the communication module of the control unit 20, linked to the concept of telemedicine.

According to an alternative embodiment, the diagnostic assistance module 28 uses biomarkers based on ULM. More precisely, the diagnostic support module can calculate biomarkers from ULM 16 data in order to refine the diagnosis and assist a non-expert operator.

Examples of these biomarkers are provided further down in the text.

When using the system 10 according to the present invention, the operator can use a module present on the device and which allows the following operations to be carried out:
connecting the control unit 20 with an expert not present using one or more of the following telecommunications methods:
   voice communication between the patient, the operator and the expert,
visual communication between the patient, the operator and the expert, remote control, by the expert:
   software,
   of the positioning assistance module 26,
exchange of data of interest, for example relevant metadata (position, environment, etc.)
exchange of relevant medical information and examination results to and from control unit 20,
assistance in establishing a remote diagnosis,
treatment proposal and/or recommendation.

In cases where the diagnosis makes it possible to establish eligibility for emergency treatment, the treatment can be carried out immediately by the operator located at the patient's bedside, on advice and in continuous consultation with the expert.

In the present application, the term “immediate” refers to a duration shorter than the return journey to the hospital, typically a few minutes.

According to a second embodiment, the diagnostic assistance module 28 makes it possible to assist expert and non-expert operators in their use of the system 10 according to the invention by means of specific software. Particularly in that ULM data is transmitted quickly by telemedicine. The control unit 20 then displays, on an additional display device, various indicators to assist the operator but does not perform a diagnosis. It provides several functionalities:
assistance with differential diagnosis,
a detailed analysis of the ultrasound image 16 obtained.

This embodiment can be achieved for example by using artificial intelligence software. Thus, it is the combination between the data from super-resolved ultrasound (ULM) and the artificial intelligence process(es)/software which makes it possible to calculate specific biomarkers, thus making it possible to help a non-expert operator with the diagnosis and consequently the mobility use of the system 10 according to the invention.

The differential diagnosis assistance makes it possible to estimate the severity and location of the problem area within the biological target structure 100, for example a lesion site. This function is based on medical practices already in place and allows the operator to carry out these operations directly on the device. Following the indications provided by the operator, the diagnostic assistance module 28 suggests probabilities of locating the problem area to allow more efficient positioning and use.

The diagnostic assistance module 28 can be coupled with the position assistance module 26 to directly enable each probe 24 to be placed so as to image a specific area.

The fine analysis of the image obtained by the system 10 allows, in one or more specific regions of the biological target structure 100 of the patient, to reprocess the data allowing the extraction of quantitative parameters. In particular, the diagnostic assistance module 28 can determine, for example in the case of use of the system 10 according to the present invention in the context of a patient presenting signs of stroke, reference medical indicators such as Cerebral Blood Volume (CBV) or Cerebral Blood Flow (CBF), which are classic imaging biomarkers. New biomarkers will also be proposed, in particular:
la topographie d'une zone particulière de la structure biologique cible 100, plus particulièrement la zone à problème et, plus précisément, dans le cas d'un AVC, de la zone d'hypoperfusion.
speed measurements in individual vessels selected by the operator,
the topography of a particular area of the biological target structure 100, more particularly the problem area and, more precisely, in the case of a stroke, of the hypoperfusion zone.

Other indications, other than hypoperfusion, are possible, among others general parameters on the paths of tracking elements 14, such as histograms of speed, direction, dispersion.

By using artificial intelligence methods, probabilities associated with the different known diagnoses can be estimated in order to guide the operator. In particular, the comparison of the ultrasound image 16 with a relevant data bank, for example of the different arterial territories in the case of a stroke, or with contralateral imaging of the other hemisphere of the patient's brain, allows to quantify the certainty with which the diagnosis (for example of ischemia) can be proposed.

In an emergency clinical context, for the particular case of a patient having suffered a stroke, and with a system 10 using ULM imaging, the system 10 according to the invention can be used, regardless of the environment in which the patient is located, as follows:
call to emergency services and arrival of emergency services equipped with the emergency imaging system 10 according to the symptoms described and the result of the patient's history for which emergency services were called,
establishment of a preliminary diagnosis based on the examination of neurological syndromes (visual impairment, aphasia, hemiparesis, etc.), and characteristic clinical manifestations already used in the state of the art,
if a stroke is suspected:
   connection of one or more probes 24 to the control unit 20,
   potential installation of an ECG on the patient and connected to the control unit 20,
   engagement of the positioning assistance module 26,
   application of gel to the patient's temples, positioning of probes 24 on the temples,
   acquisition of a first image,
   correction of the positioning of the probes 24 by means of the positioning assistance module 26, until optimization of the signal perceived by the control unit 20,
   securing of the probes 24 on the patient's head,
   evaluation of the quality of the signals before launching the ULM acquisition sequence,
   ULM ultrasound acquisition,
   injection, in the first seconds of acquisition, of tracking elements 14, more particularly of intravenous contrast agents,
   potential correction of the positioning of probes 24 with ULM data,
   creation of the ultrasound image 16 of the biological target structure 100, more particularly, of an angiography using ULM technology,
   immediate transmission of the ultrasound image 16 to a display device 18, said display device 18 being remote, the transmission allowing a neuroradiologist, a person trained in reading these ULMs (an expert) or a system expert to read the ultrasound image 16,
   establishment of a diagnosis by an expert operator or not (the neuroradiologist, the person trained to read these ULMs or the expert system, for example),
if the diagnosis confirms the presence of a thrombus, then the doctor validates the injection of thrombolytic to the patient, significantly reducing treatment time,
the patient is taken to the hospital for further diagnosis by MRI or CT scan, and is treated.

The present invention therefore proposes a mobile diagnostic system, resulting from the combination of the use of a portable imaging system 10 with a super-resolved ultrasound (ULM) method. It is precisely the synergies between the ULM and different elements of the system which form the key to mobility diagnosis.

Possibly, later, when the patient is stabilized, the system 10 according to the present invention can, for example, be used for the following applications:
regular monitoring of blood vessels and micro-vessels to determine the evolution of the patient's brain post-stroke, thus making it possible to predict, among other things, negative developments linked to vasospasm,
patient monitoring, excluding emergencies, in general.

1-14. (canceled)

15. A portable ULM imaging system for imaging a biological target structure of a patient by means of at least one tracking element introduced within said biological target structure, the portable ULM imaging system comprising:
a control unit with a memory, the memory being intended to store at least one ultrasound image characterising the biological target structure;
at least one first probe intended to be removably fixed to a biological securing structure of the patient, the biological securing structure at least partially surrounding the biological target structure, the at least one first probe being connected to the control unit; and
a positioning assisting module for the at least one probe,
the at least one first probe enabling the system to detect the at least one tracking element by means of ultrasounds, the control unit being configured to locate, in real time, the at least one tracking element inside the biological target structure, so as to implement a ULM method to characterise the biological target structure and create the ultrasound image,
the control unit further being designed to display, on a display device, the at least one ultrasound image stored in the memory, and
the ultrasound image enabling an operator to make a rapid diagnosis independently of the patient's location and environment.

16. The system according to claim 15, wherein the biological target structure is the cerebral blood system and the ultrasound image is an angiography.

17. The system according to claim 15, wherein the at least one tracking element is a contrast agent.

18. The system according to claim 15, wherein the display device is located in a different place from the control unit and/or the at least first probe.

19. The system according to claim 15, wherein the control unit and the display device communicate by electromagnetic waves.

20. The system according to claim 15, wherein the system comprises a diagnostic assistance module.

21. The system according to claim 20, wherein the diagnostic assistance module enables a first operator at the patient's bedside to be put into contact with a second operator able to read the ultrasound image displayed on the display device.

22. The system according to claim 20, wherein the diagnostic assistance module comprises software for assisting an operator at the patient's bedside.

23. The system according to claim 15, wherein the positioning assisting module is a passive mechanical module.

24. The system according to claim 15, wherein the positioning assisting module is an active electronic component.

25. The system according to claim 24, wherein the positioning assisting module calculates a transmission quality index for each probe.

26. The system according to claim 25, wherein the position assisting module (26) sends at least one feedback signal so as to help maximise the quality index.

27. The system according to claim 15, wherein the system comprises a transmission module.

28. A rapid imaging method intended to visualise a biological target structure of a patient, the method enabling the system according to claim 15 to be implemented, the method comprising the following steps, in the chronological order in which they are stated:
a) connecting, if necessary, by a first operator, of the at least one probe to the control unit,
b) engaging the positioning assistance module,
c) positioning the probes on the patient's biological securing structure,
d) acquisition of a first ultrasound image,
e) correction, if necessary, of the positioning of at least one probe by means of the positioning assistance module, until optimisation of the signal perceived by the control unit,
f) positioning of the at least one probe on the biological securing structure,
g) evaluation of the quality of the signals before launching the sequence for acquiring a new ultrasound image,
h) injecting, in the first few seconds of acquisition, of tracking elements into the patient's biological target structure,
i) acquisition of the ultrasound image,
j) correction, if necessary, of the positioning of at least one probe by means of the positioning assistance module until optimisation of the signal perceived by the control unit using ULM technology,
k) creation of the ultrasound image of the biological target structure using ULM technology,
immediate transmission of the ultrasound image to a display device within reach of a second operator.
